(19) United States
(12) Patent Application Publication
(10) Pub. No.: US 2025/0082552 A1
(43) Pub. Date: Mar. 13, 2025

(54) Dental Care Products Comprising Stannous Flouride

(71) Applicant: Credentis AG, Windisch (CH)

(72) Inventors: Divya Devidas Naik, Baar (CH); Marzia Massignani, Baar (CH); Haleh Abivardi Brönner, Baar (CH); Golnar Abivardi Signer, Baar (CH); Michael Hug, Baar (CH); Dominikus Amadeus Lysek, Baar (CH)

(21) Appl. No.: 18/464,817

(22) Filed: Sep. 11, 2023

(51) Int. Cl. A61K 8/21; A61K 8/64; A61Q 11/00

(52) U.S. Cl. CPC A61K 8/21; A61K 8/64; A61Q 11/00; A61K 2800/92

(57) ABSTRACT

The present invention provides new dental care products comprising stannous fluoride and at least 0.01 wt % self-assembling peptides, wherein the self-assembling peptides are essentially present in the dental care product in assembled form. The assembled peptides form a three-dimensional matrix capable of stabilizing stannous fluoride by protecting it from oxidation. The dental care products may further comprise polyphosphates such as sodium tripolyphosphates as well as a tin chelating agent such as sodium gluconate. They may be provided in the form of a dental gel, a toothpaste, a tooth foam, dental rinse or a prophylactic paste. Also disclosed are methods for stabilizing stannous fluoride in a dental care product. Finally, the present invention relates to therapeutic methods for protecting a tooth from demineralization, for remineralizing the tooth enamel of a non-cavitated tooth lesion, for destroying biofilm formed on tooth and for treating dentinal hypersensitivity.

This application contains a sequence listing filed in ST.26 format entitled “320406-1300 Sequence Listing” created on Sep. 11, 2023 and having 28,487 bytes. The content of the sequence listing is incorporated herein in its entirety.

The present invention provides new dental care products comprising stannous fluoride and at least 0.01 wt % self-assembling peptides, wherein the self-assembling peptides are essentially present in the dental care product in assembled form. The assembled peptides form a three-dimensional matrix capable of stabilizing stannous fluoride by protecting it from oxidation. The dental care products may further comprise polyphosphates such as sodium tripolyphosphates as well as a tin chelating agent such as sodium gluconate. They may be provided in the form of a dental gel, a toothpaste, a prophylactic paste, a tooth foam or a dental rinse. Also disclosed are methods for stabilizing stannous fluoride in a dental care product. Finally, the present invention relates to therapeutic methods for protecting a tooth or teeth from demineralization, for remineralizing tooth enamel, for destroying biofilm formed on a tooth or teeth and for treating dentinal hypersensitivity.

The anti-caries effects of fluoride are scientifically demonstrated and thus well-established. Fluoride inhibits tooth demineralization and promotes tooth remineralization by reacting with calcium present in plaque and saliva to form calcium fluoride on the tooth surface. The calcium fluoride eventually dissolves to allow calcium and fluoride ions to interact with the tooth and to form fluoride-containing apatite within the tooth structure. By converting the calcium mineral apatite in teeth into fluorapatite, fluoride also makes the tooth enamel more resistant to bacteria-generated acid attacks. Moreover, fluoride can also directly inhibit plaque bacteria by interfering with the bacterial metabolism through various different mechanisms (https://en.wikipedia.org/wiki/Tin (II)_fluoride; Marquis, 1995; Nasser et al., 2023).

In light of these known advantageous effects of fluoride, dental care products such as toothpastes (dentrifices) comprising fluoride are nowadays commonly in use.

Historically, the first fluoride-containing dentifrices provided the fluoride in the form of tin(II) fluoride, also commonly referred to as stannous fluoride (SnF₂). These stannous fluoride-comprising toothpastes exhibited considerable efficacy in reducing coronal caries; however, the use of stannous fluoride in oral care compositions was limited due to its instability in the presence of water. The stannous ions (Sn²⁺) are highly reactive and rapidly oxidate into inactive stannic ions (Sn⁴⁺). Oxidation of Sn²⁺ manifests itself intraorally as yellow-brown stains on the tooth surface. Moreover, the tin present in stannous fluoride readily hydrolyses above a pH of 4, which results in its precipitation from the solution, with a consequent loss of its therapeutic properties. Importantly, the chemical reactions contributing to stannous fluoride deactivation occur not only in storage, but directly in processing (White, 1995).

In consequence, manufacturers of fluoride-containing toothpaste products switched to using alternative fluoride sources such as, e.g., monofluorophosphate or sodium fluoride, which exhibit anticaries activities comparable to those of the fluoride ion in stannous fluoride. However, unlike monofluorophosphate or sodium fluoride, stannous fluoride is also known for its intrinsic antimicrobial effects and its clinically demonstrated efficacy in controlling gingivitis. Moreover, it shows significant reactivity with root surfaces and was demonstrated to reduce dentinal hypersensitivity. Accordingly, in recent years there has been renewed interest in the manufacture of dental products containing stannous fluoride, as stannous fluoride is considered a therapeutically effective “all-in-one” active ingredient (White, 1995).

Therefore, scientists were faced with the challenge of finding ways to stabilize stannous fluoride in dental care products and during administration, as stabilized stannous fluoride formulations allow for greater bioavailability of stannous and fluoride ions, thereby increasing their oral health benefits. Stabilization of stannous fluoride requires prevention of Sn²⁺ oxidation into Sn⁴⁺ as well as protection of SnF₂ from hydrolysis. As it turned out, earlier stannous fluoride-containing dentifrices were oftentimes stable enough to provide some anticaries efficacy but provided insufficient bioavailable stannous fluoride to exert antimicrobial activity required for gingivitis control. In other words, the stability requirements of stannous fluoride as gingivitis active agent appeared to be more rigorous than those for anticaries activity (White et al., 1995).

Over time, a number of strategies have been developed to obtain more stable stannous fluoride-containing formulations:

For instance, Sn²⁺ oxidation to stannic ions may be prevented, e.g., by drastically reducing or completely omitting the addition of water to the SnF₂-containing toothpaste formulations. However, a low water content of less than 10% can affect the taste and texture of the resulting toothpaste.

The stannous fluoride may also be dissolved in an anhydrous material such as glycerin to obtain non-aqueous gel formulations. For instance, U.S. Pat. Nos. 4,418,057A and 3,433,544A describe methods where stannous fluoride is formulated as a non-aqueous gel mixture comprising anhydrous glycerin and hydroxyethyl cellulose as a gelling agent.

Further attempts to stabilize stannous fluoride in dental care products include the addition of stannous salts capable of reacting with oxygen to protect the stannous fluoride from becoming oxidized. Stannous salts such as stannous phosphate or stannous chloride may also provide a reservoir source for replenishing Sn²⁺ lost from the active SnF₂. U.S. Pat. No. 5,004,597A discloses an exemplary composition containing stannous fluoride that relies on stannous chloride and stannous gluconate as stannous reservoirs for replacing unstable Sn²⁺ ions. However, the use of some of these stannous salts may negatively affect the taste of the toothpaste and, more importantly, has proven to be rather ineffective in preventing the formation of high amounts of stannic ions.

Use of chelating agents has also been contemplated to protect stannous fluoride complexes from hydrolysis and oxidation and thus to reduce or even eliminate the precipitation of insoluble tin compounds. Suitable stannous chelants include sodium gluconate, stannous gluconate, citrates such as zinc citrate as well as hydroxyl-substituted aliphatic di- and tri-carboxylic acids such as citric acid or malic acid. Formulations comprising these chelating agents are disclosed, e.g., in U.S. Pat. No. 5,716,600A, 5,004,597A, 9,968,803B2 or 3,282,792A. Alternatively, a copolymer of maleic anhydrate or acid and a polymerizable ethylenically unsaturated monomer, preferably a lower alkyl vinyl ether such as methoxyethylene has been found to form chelates with Sn²⁺, thereby protecting the Sn²⁺ ions from oxidation. The use of such polymeric chelating agents in stannous fluoride-containing formulations is disclosed, e.g., in U.S. Pat. No. 5,017,363A, 4,960,586A or 4,961,924A. However, inclusion of some of the above-mentioned chelants can result in toothpastes with a gritty texture and a bitter, metallic, stringent, and salty taste profile (Li et al., 2019).

Finally, Colgate Total SF manufactured by the Colgate-Palmolive Company relies on a formulation in which SnF₂ is stabilized using zinc phosphate, which complexes with SnF₂ to prevent its oxidation.

Despite the different approaches and strategies contemplated for stabilizing stannous fluoride in dental care formulations, there remains a need for improved stannous fluoride-containing dental care products that are characterized both by a stable formulation and by an increased efficacy.

The problem is solved by the present invention, in particular by the subject-matter of the present claims.

The present invention provides a dental care product comprising:
i. tin(II) fluoride, and
ii. at least 0.01 wt % self-assembling peptides comprising a sequence of X1-X2-X1-X2-X1, wherein X1 is independently selected from the group consisting of glutamic acid, aspartic acid, glutamine and ornithine, and X2 is independently selected from the group consisting of alanine, valine, isoleucine, leucine, methionine, phenylalanine, tyrosine, tryptophan and glutamine, wherein the self-assembling peptides are essentially present in the dental care product in assembled form.

The dental care product of the invention comprises tin(II) fluoride, which, in the context of the present invention, is also referred to as stannous fluoride or SnF₂. The stannous fluoride may be present in the dental care product at a concentration of 0.1-1.0 wt %.

Preferably, the stannous fluoride is present in the dental care product at a concentration of 0.2-0.7 wt %. Previous research has demonstrated that stannous fluoride at a concentration of 0.4-0.7 wt % (corresponding to about 1000 to 1500 ppm fluoride), in particular of 0.454 wt %, provides the best therapeutic protection against caries, gingivitis, plaque and dentin sensitivity (West et al., 2018; Parkinson et al., 2020). For children, the recommended concentration is 0.227 wt % (about 500 ppm fluoride) Thus, in a preferred embodiment, the dental care product of the invention comprises stannous fluoride at a concentration of 0.1-0.7 wt %, most preferably at a concentration of 0.454 wt %.

The inventors surprisingly found that the dispersion of stannous fluoride in a three-dimensional matrix formed by self-assembling peptides effectively protects stannous ions (Sn²⁺) from being oxidized to stannic ions (Sn⁴⁺), thus preventing the formation of extrinsic stains on the tooth surface caused by said oxidation.

In the context of the invention, self-assembling peptides (SAP) are peptides that, as the name implies, are capable of undergoing self-assembly in a pH-dependent manner to form three-dimensional scaffolds or matrices, thereby promoting tissue regeneration. As used herein, “self-assembly” of the peptides refers to the spontaneous and reversible organization of peptides with other peptides of their own kind (or peptides having a similar structure) into multimeric assemblies by non-covalent interactions. Non-covalent interactions that are responsible for forming the multimeric assemblies include van-der-Waals, pi-stacking, hydrogen bonds, polar and ionic interactions between the amino acid backbones and/or the amino acid side chains of the peptides.

Suitable self-assembling peptides are taught, e.g., in WO 2004/007532 A1, U.S. Ser. No. 10/521,628, U.S. Ser. No. 12/729,046, U.S. Ser. No. 13/551,878, U.S. Ser. No. 14/062,768, WO 2010/041636 A1 or WO2014/027012 A1, which are all fully incorporated herein by reference. Self-assembling peptides can assemble in one dimension to form beta-sheets, and higher order assemblies such as fibers or tape-like assemblies. Three-dimensional supramolecular structures of self-assembling proteins can be formed, which have an affinity for/to calcium phosphate.

The size of the self-assembling peptides used in the dental care product of the invention is not specifically limited. They may be of any length that allows self-assembly in a pH-dependent manner. The peptides may have a size of about 5-200 amino acids, e.g., 9-100 amino acids, 10-50 amino acids, 10-30 amino acids or 11-20 amino acids. Preferably, the self-assembling peptides have a length of about 27 amino acids, 24 amino acids, 21 amino acids, 15 amino acids, or 11 amino acids. In a particularly preferred embodiment, the self-assembling peptides have a length of 11 amino acids, i.e., they consist of 11 amino acids.

To be able to adhere to a tooth surface, the matrix formed by the self-assembling peptides present in the dental care product of the invention has to be able to bind mineral particles present in the tooth surface. The matrix thus comprises binding sites for the mineral particles which enable it to bind the particles, which preferably comprise calcium, on the tooth surface. For example, charged amino acid residues such as Glu on the surface of self-assembling peptides bind to hydroxyapatite particles and to the tooth surface, which is also substantially formed of hydroxyapatite. A capability for three-dimensional self-organization is important for binding. In general, highly charged surfaces will promote adhesion of the mineral particles. The protein-matrices work particularly well when their surface shows glutamate residues which may attach to calcium phosphate or to other mineral particles. Preferably, the protein comprises 5% or more, 10% or more, 20% or more or 30% or more charged amino acid residues, such as glutamate residues.

In the context of the present invention, self-assembling peptides may be able to self-assemble by themselves, as is the case, e.g., for the peptides P11-4, P11-8, P11-2, P11-5 mentioned below, but they can alternatively be able to self-assemble in a combination of two self-assembling peptides, as is the case, e.g., for the peptides P11-13/P11-14 and P11-28/P11-29, P11-30/P11-31 mentioned below.

Self-assembling peptides in the dental care product of the invention comprise the consensus sequence X1-X2-X1-X2-X1, wherein X1 is independently selected from the group consisting of glutamic acid, aspartic acid, glutamine and ornithine, and X2 is independently selected from the group consisting of alanine, valine, isoleucine, leucine, methionine, phenylalanine, tyrosine, tryptophan and glutamine. Independently selected means that, e.g., X1 in positions 1, 3 or 5 of the sequence above can be different from each other. Of course, they can also be identical.

In one embodiment, the self-assembling peptides comprise the sequence of X1-X2-X1-X2-X1, wherein X1 is an amino acid with an acidic side chain and X2 is an amino acid with a hydrophobic or polar side chain.

Preferably, the self-assembling peptides also comprise X1-X2-X1-X2-X1, wherein X1 is independently selected from the group consisting of glutamic acid and ornithine, and X2 is independently selected from the group consisting of tryptophan and phenylalanine.

Self-assembling peptides may further comprise X3-F-X1-W-X1-F-X1, wherein X1 is independently selected from the group consisting of glutamic acid and ornithine, and X3 is selected from the group consisting of arginine, glutamic acid and ornithine, wherein X3 preferably is arginine.

Self-assembling peptides used in the dental care product the invention may comprise or, preferably, consist of X4-X4-X3-F-X1-W-X1-F-X1-X4-X4, wherein X1 is independently selected from the group consisting of glutamic acid and ornithine, and wherein X3 is selected from the group consisting of arginine, glutamic acid and ornithine, and wherein X4 is independently selected from the group consisting of glutamine, glutamic acid, serine, threonine and ornithine. X3 preferably is arginine. Independently, X4 preferably is glutamine.

Self-assembling peptides may comprise SEQ ID NO: 5, or, preferably, consist thereof: Q-QR-F-X1-W-X1-F-X1-Q-Q, wherein X1 is independently selected from the group consisting of glutamic acid and ornithine.

In the context of the present invention, self-assembling peptides taught in WO 2004/007532 A1, U.S. Ser. No. 10/521,628, U.S. Ser. No. 12/729,046, U.S. Ser. No. 13/551,878, U.S. Ser. No. 14/062,768, or WO2014/027012 A1, which are all fully incorporated herein by reference, are preferred. Most preferably, said peptides comprise the specific peptides listed in Table 2 or consist thereof. Of course, self-assembling peptides assembling in combination with another self-assembling peptide, e.g., as disclosed above, may be formulated in composition.

Preferably, the self-assembling peptide comprises the sequence of SEQ ID NO: 6 or consists thereof. A peptide consisting of a sequence of SEQ ID NO: 6 is also designated P11-4, and is preferred throughout the invention. In another preferred embodiment, the self-assembling peptide comprises the sequence of SEQ ID NO: 9 or consists thereof (P11-8). In another preferred embodiment, the self-assembling peptide comprises the sequence of SEQ ID NO: 15 or consists thereof (P11-20).

Peptides of SEQ ID NO: 6, 9 or 15 are particularly advantageous, e.g., as they can be used in relatively low concentrations, they are highly compatible with cells and have beneficial charge distribution.

TABLE 1
Consensus sequences of preferred self-assembling peptides

| SEQ ID | Peptide | | |
|---|---|---|---|
| NO | name | Sequence | Exemplary SAP |
| SEQ ID | Consensus | X1-X2-X1-X2-X1, | P11-2, P11-4, |
| NO: 1 | sequence | wherein X1 is independently | P11-5, P11-8, |
| | 1 | selected from the group | P11-12, P11-13, |
| | | consisting of glutamic acid, | P11-14, P11-17, |
| | | aspartic acid, glutamine and | P11-19, P11-20, |
| | | ornithine, and X2 is | P11-28, P11-29 |
| | | independently selected from | |
| | | the group consisting of | |
| | | alanine, valine, isoleucine, | |
| | | leucine, methionine, | |
| | | phenylalanine, tyrosine, | |
| | | tryptophan and glutamine | |
| SEQ ID | Consensus | X1-X2-X1-X2-X1, | P11-4, P11-8, |
| NO: 2 | sequence | wherein X1 is independently | P11-12, P11-13, |
| | 2 | selected from the group | P11-14, P11-17, |
| | | consisting of glutamic acid | P11-28, P11-29 |
| | | and ornithine, | |
| | | and X2 is independently | |
| | | selected from the group | |
| | | consisting of tryptophan and | |
| | | phenylalanine | |
| SEQ ID | Consensus | X3-F-X1-W-X1-F-X1, | P11-4, P11-8, |
| NO: 3 | sequence | wherein X1 is independently | P11-12, P11-13, |
| | 3 | selected from the group | P11-14, P11-17, |
| | | consisting of glutamic acid | P11-28, P11-29 |
| | | and ornithine, and X3 is | |
| | | selected from the group | |
| | | consisting of arginine, | |
| | | glutamic acid and ornithine, | |
| | | wherein X3 preferably | |
| | | is arginine | |
| SEQ ID | Consensus | X4-X4-X3-F-X1-W-X1-F- | P11-4, P11-8, |
| NO: 4 | sequence | X1-X4-X4, | P11-12, P11-13, |
| | 4 | wherein X1 is independently | P11-14, P11-17, |
| | | selected from the group | P11-28, P11-29 |
| | | consisting of glutamic acid | |
| | | and ornithine, | |
| | | and wherein X3 is selected | |
| | | from the group consisting of | |
| | | arginine, glutamic acid and | |
| | | ornithine, | |
| | | and wherein X4 is | |
| | | independently selected from | |
| | | the group consisting of | |
| | | glutamine, glutamic acid, | |
| | | serine, threonine and ornithine. | |
| | | X3 preferably is arginine. | |
| | | Independently, X4 preferably | |
| | | is glutamine. | |
| SEQ ID | Consensus | Q-Q-R-F-X1-W-X1-F-X1- | P11-4, P11-8 |
| NO: 5 | sequence | Q-Q, wherein independently | |
| | 5 | selected from the group | |
| | | consisting of glutamic acid | |
| | | and ornithine. | |

TABLE 2
Preferred self-assembling peptides. Positions X1
are underlined, “O” denotes the amino acid
ornithine.

| | | | % amino acid |
|---|---|---|---|
| | | | identity to |
| | | Sequence | P11-4 (ClustalW |
| | Peptide | (One | (2.1, standard |
| SEQ ID NO | name | letter code) | parameters)) |
| SEQ ID NO: 6 | P11-4 | QQRFEWEFEQQ | 100 |
| SEQ ID NO: 7 | P11-2 | QQRFQWQFEQQ | 81.8 |
| SEQ ID NO: 8 | P11-5 | QQRFOWOFQQQ | 72.7 |
| SEQ ID NO: 9 | P11-8 | QQRFOWOFEQQ | 81.8 |
| SEQ ID NO: 10 | P11-12 | SSRFOWOFESS | 45.4 |
| SEQ ID NO: 11 | P11-13 | EQEFEWEFEQE | 72.7 |
| SEQ ID NO: 12 | P11-14 | QQOFOWOFOQQ | 63.6 |
| SEQ ID NO: 13 | P11-17 | TTRFEWEFETT | 63.6 |
| SEQ ID NO: 14 | P11-19 | QQRQOQOQEQQ | 54.5 |
| SEQ ID NO: 15 | P11-20 | QQRQEQEQEQQ | 72.7 |
| SEQ ID NO: 16 | P11-28 | OQOFOWOFOQO | 45.4 |
| SEQ ID NO: 17 | P11-29 | QQEFEWEFEQQ | 90.9 |
| SEQ ID NO: 18 | P11-16 | NNRFOWOFENN | 45.4 |
| SEQ ID NO: 19 | P11-18 | TTRFOWOFETT | |
| SEQ ID NO: 20 | P11-26 | QQOQOQOQOQQ | 36.4 |
| SEQ ID NO: 21 | P11-31 | SSOFOWOFOSS | 27.3 |

The composition of the invention may also comprise at least one self-assembling peptide having at least 45% sequence identity to a peptide consisting of SEQ ID NO: 6. Preferably, the peptide has at least 54%, at least 63%, at least 72%, at least 81% or at least 90% sequence identity to a peptide consisting of SEQ ID NO: 6, or is said peptide.

The self-assembling peptides used in the dental care products of the invention may further be structurally modified in one or more amino acid positions, e.g., by the introduction of one or more modified amino acids. According to the invention, these modified amino acids may be amino acids that have been changed by e.g., biotinylation, phosphorylation, glycosylation, acetylation, branching and/or cyclization. Further, the self-assembling peptides present in the dental care product of the invention may additionally or alternatively contain other modifications, such as terminal blocking groups, formyl-, gamma-carboxyglutamic acid hydroxyl-, methyl-, phosphoryl-, pyrrolidone carboxylic acid-, and/or sulphate-groups.

In a preferred embodiment, all self-assembling peptides present in the dental care product of the invention are acetylated at their N-terminus and/or amidated, e.g., with an NH₂-group, at their C-terminal end, most preferably, both. As non-blocked forms tend to start a deaminization reaction, the termini of the self-assembling peptides described herein are preferably blocked to increase stability. Accordingly, in a preferred embodiment, the self-assembling peptides present in the dental care product of the invention, e.g., peptides of SEQ ID NO: 6, 9 and/or 15, comprise both an Ac-N-terminus and NH₂-C-Terminus. A particularly preferred embodiment is a peptide P11-4 (SEQ ID NO: 6) that is N-terminally acetylated and C-terminally amidated with a NH₂-group.

The dental care product may contain a single type of self-assembling peptides or two or more, such as three, four or five etc., different types of self-assembling peptides, e.g., it may comprise a mixture of self-assembling peptides consisting of SEQ ID NO: 6 and self-assembling peptides consisting of SEQ ID NO: 15.

In the dental care product of the invention, the self-assembling peptides are essentially present in assembled form. As used herein, this means that at least about 70%, about 75%, about 80%, about 85%, about 90%, about 95%, about 99% or even about 100% of the peptides found in the dental care product are assembled i.e., in multimeric or polymeric form.

Most self-assembling peptides in the dental care product according to the invention, e.g., P11-4 (SEQ ID NO:6) and/or P11-20 (SEQ ID NO: 15) as well as terminally modified variants thereof undergo self-assembly as soon as the pH of their environment drops below pH 7.5. This means that the self-assembling peptides present in the dental care product of the invention start to self-assemble to a significant extent when the pH drops below 7.5.

Assembly is mainly controlled by the pH. The pH at which the self-assembling peptides assemble depends on its sequence. For example, P11-4 assembles below pH 7.5. The pH of the dental care product of the invention for P11-4 and similar peptides preferably is below 7.5, in particular, if the self-assembling peptide is P11-4. The pH preferably is between 4 and 7.5, e.g., 5-7.5 or 6-7.

Other self-assembling peptides, such as P11-8, self-assemble above pH 7.5. If such self-assembling peptides are employed, the pH of the dental care product is suitably adapted, i.e., the pH is above the pH at which the self-assembling peptides assemble., e.g., above pH 7.5.

The assembly state of peptides is also influenced by the ionic strength. The ionic strength of a solution is a function of the concentration of all ions present in that solution. Thus, even at a pH above the pH at which the peptide starts to undergo self-assembly, i.e., when the peptide is substantially monomeric in solution, a particularly high ionic strength is able to trigger the assembly of the peptide.

The skilled person will know how to determine and measure the ionic strength of a solution. The ionic strength I is generally calculated according to the formula I=½Σzi2bi, wherein z is the valence factor and bi is the molality [mol/kg {H2O}] of the ion concentration. The summation, Σ, is taken over all ions in a solution. For example, the ionic strength of a 150 mM NaCl solution is approximately 0.15 mol/L. This is also approximately the ionic strength of blood. The ionic strength of saliva present in the oral cavity, is generally much lower, such as approximately 0.04 mol/L. In the context of the invention, ionic strength in the physiological range is considered to be corresponding to an ionic strength of 0.15 mol/L.

It is also known to the skilled person that the peptide concentration may influence the assembly of peptides, i.e., a particularly high peptide concentration may trigger assembly. Correspondingly, an exceptionally low peptide concentration may prevent assembly of the peptides of the invention, i.e. even under low pH conditions as present in tooth lesions and the oral cavity.

The skilled person will be able to determine whether essentially all of the self-assembling peptides are in an assembled form by means of routine experimentation. For example, the assembly state of the peptides in solution can be determined by nuclear magnetic resonance (NMR), such as 1H-NMR, by circular dichroism analysis, by dynamic light scattering (DLS) analysis, diffusing-wave spectroscopy, native electrophoretic methods, viscosity measurements (rheology), Quartz crystal microbalance with dissipation monitoring (QCMD) and the like, preferably by native electrophoretic methods. The presence of fibres of self-assembled peptide may be detected by TEM.

Suitable buffers and pH modulating agents for obtaining the desired pH are known in the art.

As described herein, stabilization of stannous fluoride involves prevention of premature oxidation of stannous ions (Sn²⁺) to stannic ions (Sn⁴⁺). The inventors surprisingly found that the dispersal of stannous fluoride in a three-dimensional matrix formed by the herein described self-assembling peptides protects the stannous ions from being oxidated within the dental care product and upon application of the dental care product to a dental surface. Without being bound by theory, the fibres formed by the self-assembling peptides, having a net-charge of −2, may serve as a stabilization factor, thereby preventing the oxidation of the stannous ion. The three-dimensional matrix of self-assembling peptides, through its affinity to hydroxyapatite, further helps to maintain stannous fluoride on the enamel surface for prolonged durations. The added stabilization of the stannous ion due to the presence of the self-assembling peptides prevents the formation of dental staining typically observed as a result of SnF₂ oxidation.

Of note, the present inventors found that the surprising ability of the herein described self-assembling peptides to stabilize and protect stannous fluoride from oxidative changes largely depends on the concentration of the self-assembling peptides within the dental care product: The advantageous affect was only observable in formulations containing 100 PPM (i.e. 0.01 wt %) or more of self-assembling peptides whereas this effect was not noticeable in formulations having a concentration of self-assembling peptide of less than 100 PPM.

Accordingly, the self-assembling peptides are present in the dental care product of the invention at a concentration of at least 100 PPM or at least 0.01 wt %, e.g., at a concentration of 0.01-5 wt % such as 0.01-0.1 wt %, 0.1-1 wt %, 1-2 wt %, 2-3 wt %, 3-4 wt % or 4-5 wt %. Preferably, the self-assembling peptides are present in the dental care product at a concentration of 0.01-0.1 wt %, e.g., about 0.01, 0.02, 0.03, 0.04, 0.05, 0.06, 0.07, 0.08, 0.09 or 0.1 wt %. Most preferably, the self-assembling peptides are present in the dental care product at a concentration of about 0.03-0.07 wt %, e.g., about 0.05%.

While it is not required, in a preferred embodiment, the dental care product of the invention further comprises a polyphosphate. Polyphosphates are inorganic polymers consisting of two or more phosphate molecules arranged primarily in a linear configuration, although in some cases, cyclic derivatives may be used as well. Polyphosphates act as chelators and are capable of preventing dental pellicle formation by binding to the tooth surface. The polyphosphates thereby reduce the force of adhesion of absorbed salivary glycoproteins and help desorb salivary proteins from enamel (Mason et al., 2019). In consequence, polyphosphates contribute to stain removal and the control of stain build-up during tooth brushing. Accordingly, polyphosphate compounds are frequently used in whitening toothpastes. Moreover, due to their ability to act as chelating agents, polyphosphates can bind to calcium ions in saliva and plaque, thereby suppressing the formation of calcified deposits (known as calculus) on dental surfaces.

Surprisingly, the inventors have found that polyphosphates do not prevent the adhesion of the self-assembling peptides to the enamel, and they can thus be combined in a dental care product without a loss of the dental effects of self-assembling peptide.

The Food and Drug Administration (FDA) has listed sodium pyro-, tri-, and hexametaphosphates as Generally Recognized as Safe (GRAS) food additives (Moon et al., 2019). Any of these, or a combination thereof, can be used in the dental care product of the invention.

Dental care formulations comprising polyphosphates such as, e.g., sodium hexametaphosphate for reducing stains arising from oxidation of stannous fluoride have been disclosed e.g., in U.S. Pat. No. 6,667,027B2, 6,350,436B1, 6,821,507B2, 5,578,293A, 5,145,666A, 5,281,411A or 5,281,410A.

In some embodiments, the polyphosphate present in the dental care product of the invention may be a pyrophosphate, i.e., a diphosphate characterized by two phosphorous atoms linked to one oxygen atom. The pyrophosphate may be provided in the dental care product as a disodium or tetrasodium salt.

However, in a preferred embodiment, the polyphosphate present in the dental care product of the invention is sodium tripolyphosphate (STP). The STP may be present in the dental care product at a concentration of 1-10 wt %, e.g. 2-5 wt %. Preferably, the dental care product of the invention comprises about 3 wt % STP.

The dental care product optionally further comprises additional ingredients known for their ability to stabilize stannous fluoride.

For instance, in some embodiments, the oral care product may further comprise one or more agents capable of chelating stannous ions. Chelation refers to a chemical reaction in which ions or molecules bind to metal ions. Suitable stannous chelating agents capable of forming stable chelates with stannous fluoride and thus protecting it from hydrolysis and oxidation are known in the state of the art and include, e.g., sodium gluconate, stannous gluconate or zinc citrate.

The agent capable of chelating stannous ions may be present in the dental care product at a concentration of 0-5 wt %, e.g., 0.5-1 wt %, 1-2 wt %, 2-3 wt %, 3-4 wt % or 4-5 wt %. More preferably, the agent capable of chelating stannous ions is present in the dental care product at a concentration of 2-4 wt %, e.g., 2 wt %, 3 wt %, or 4 wt %.

Preferably, the agent capable of chelating stannous ions is sodium gluconate. Sodium gluconate is non-corrosive, non-toxic and readily biodegradable. It forms stable chelates with calcium, iron, copper, aluminium and other heavy metals, including tin. Thus, sodium gluconate is preferably used in the dental care product of the present invention to further stabilize stannous fluoride.

Thus, in a preferred embodiment, the present invention provides a dental care product comprising:
i. 0.1-1.0 wt % tin(II) fluoride;
ii. at least 0.01 wt % self-assembling peptides consisting of SEQ ID NO: 6 (P11-4);
iii. 1-10 wt % sodium tripolyphosphate; and
iv. optionally, 0.5-5 wt % sodium gluconate,
   wherein the pH of the dental care product is below 7.5.

The dental care product according to the invention may be a dental gel, a toothpaste (dentifrice), a prophylactic paste, a tooth foam or a dental rinse, preferably a dental gel. The dental care product may be for use by a professional in a dental practice, where it is preferably applied using a suitable dental instrument such as rubber polisher. It can also be applied with a syringe. The dental care product may however also be for home use, where it is preferably applied to the dental surface using a finger or a toothbrush, e.g., an interdental toothbrush.

The dental care product may additionally comprise one or more typical ingredients of the respective dental care product. Such typical ingredients may be:
abrasive agents such as carbonates, phosphates, silicates, acrylates, alumina,
suspension agents such as glycerine, polyethylene glycols (PEG), sorbitol, xylitol, and/or erythritol,
binding agents such as cellulose and derivate thereof such as carboxymethyl cellulose (cellulose gum), carrageenan, paraffin, xylose,
detergents such as hydrogenated castor oil, sodium lauryl sulphate,
aroma such as caramel, vanillin, menthol,
conserving agents such as ethanol, sodium benzoate,
colouring agents such as solvent red, acid blue 3,
additional fluoride sources, preferably, in the form of tertiary amines, such as amine fluoride or organic fluoride such as sodium monofluorophosphate.

The dental care product typically comprises water, optionally, more than 10% of water.

For instance, a particular preferred dental care product of the invention comprising stannous fluoride and the herein described self-assembling peptides is a dental gel and further comprises
a. sodium tripolyphosphate,
b. sodium gluconate,
C. sorbitol,
d. cellulose gum,
e. xylitol,
f. erythritol
g. hydrated silica,
h. phosphoric acid,
i. optionally, NaOH, and
j. water

The present invention provides dental care products in which stannous fluoride is stabilized using a matrix formed by the herein described self-assembling peptides. Accordingly, the present invention also relates to a novel method for stabilizing stannous fluoride in a dental care product, comprising admixing
i) tin(II) fluoride, and
ii) at least 0.01 wt % self-assembling peptides comprising a sequence of X1-X2-X1-X2-X1, wherein X1 is independently selected from the group consisting of glutamic acid, aspartic acid, glutamine and ornithine, and X2 is independently selected from the group consisting of alanine, valine, isoleucine, leucine, methionine, phenylalanine, tyrosine, tryptophan and glutamine, wherein the self-assembling peptides are essentially present in the dental care product in assembled form,
   wherein, preferably, the dental care product is a dental care product of the present invention.

The dental care product of the invention may be stable for at least 6 months at room temperature (20° C.), at least 12 months at room temperature, at least 18 months at room temperature, at least 24 months at room temperature or at least 36 months at room temperature. In this context, stable means that no visible discoloration due to oxidation of stannous fluoride has occurred.

The use of the dental care products as described herein is associated with a series of advantageous effects.

For instance, the dental care product of the invention may be used for preventing demineralization of a tooth or for remineralizing the tooth enamel, e.g., of a non-cavitated tooth lesion.

Dental caries is one of the most ubiquitous bacterial infections in the world. It is a breakdown of tooth material due to bacterial metabolites, mainly acids made by bacteria when they break down food debris or sugar on the surface or in the biofilm of the teeth. This leads to an imbalance between demineralisation and remineralisation processes. Hard tooth structures, i.e., enamel, dentin and cementum, are damaged by ongoing demineralisation, which results in carious lesions and eventually in the appearance of caries cavities. The earliest sign of a new carious lesion is the appearance of a chalky white spot on the surface of the tooth, a so-called white spot lesion (also designated an incipient carious lesion), i.e., a subsurface lesion. As the demineralisation progresses, the mineralised surface of the lesion (partially) collapses and breaks and a microcavity or a cavity, a hole in the tooth, appears. This is referred to as a (partially) cavitated carious lesion or cavitated carious lesion.

Stannous fluoride is known for its considerable efficacy in reducing coronal caries both by preventing enamel demineralization and enabling enamel remineralization (Fiorillo et al., 2020). On the other hand, as disclosed, e.g., in international patent application WO 2021/110923 A1, a matrix formed by the self-assembling peptides as described herein can constitute a protective layer or film on teeth on which it is administered. This protective layer formed by the assembled self-assembled peptides can effectively prevent further demineralisation and protects the dental enamel from acid attacks.

In addition, a matrix formed by the herein disclosed self-assembling peptides such as P11-4 can lead to an increased mineralisation (Soares et al., 2017). A self-assembling peptide matrix was further found to prevent artificial caries lesions and lead to remineralisation of enamel around orthodontic brackets (Jablonsky-Momeni et al., 2019).

While stannous fluoride and self-assembling peptides both exert protective effects against enamel demineralization as well as remineralizing activities, they may act synergistically. Moreover, by being suspended in a three-dimensional matrix formed by the self-assembling peptides disclosed herein, stannous fluoride is maintained and stabilized on the enamel surface for extended time periods, which leads to sustained protection against tooth demineralization and prolonged enamel remineralization.

Thus the present invention provides methods for preventing demineralization of a tooth and for remineralizing the tooth enamel, e.g. of a non-cavitated tooth lesion, in a subject in need thereof, wherein both methods comprise applying an effective amount of the dental care product of the present invention to a tooth. Of course, treatment of a tooth can also be treatment of several teeth, preferably, of all teeth of a subject.

In another embodiment, the dental care product of the invention may be for destroying a bacterial biofilm formed on a tooth.

Stannous fluoride is also known for intrinsically exhibiting anti-bacterial effects both through bactericidal mechanisms as well as via the inhibition of bacterial metabolic enzymes. In line with this, the efficacy of stannous fluoride in gingivitis control has been clinically demonstrated (Paraskevas et al., 2006; Parkinson et al., 2020; Acherkouk et al., 2021). The present inventors found that the anti-bacterial effect of stannous fluoride can be further enhanced by the presence of the self-assembling peptides, which, upon self-assembly, form a negatively charged three-dimensional matrix that repels bacteria with a net negative charge. Consequently, the biofilms formed on dental surfaces are less dense and can be disrupted by stannous fluoride more easily. Therefore, the dental care product according to the present invention allows for more effective removal of dental biofilms.

Thus, the herein disclosed invention further provides a method for reducing or destroying biofilm formed on a tooth, comprising applying an effective amount of the dental care product of the invention to a tooth.

Enhanced destruction of biofilms on dental surfaces can contribute to the prevention of gingivitis, periodontitis and/or peri-implantitis.

Gingivitis is a non-destructive periodontal disease that most commonly forms as plaque-induced gingivitis in response to bacterial biofilms (also called plaque) adherent to tooth surfaces. Gingivitis is reversible with good oral hygiene.

However, in the absence of treatment, or if not controlled, gingivitis can progress to periodontitis. Periodontitis—or periodontal disease—is a set of inflammatory diseases affecting the periodontium, i.e., the tissues that surround and support the teeth. Periodontitis is caused by microorganisms that adhere to and grow on the tooth's surfaces, along with an over-aggressive immune response against these microorganisms. With the destruction of the gingival fibers, the gum tissues separate from the tooth, leading to deepened sulcus, called a periodontal pocket. Subgingival micro-organisms, i.e., those that exist apically from gum line, colonize the periodontal pockets and cause further inflammation in the gum tissues and progressive bone loss. If left undisturbed, microbial plaque calcifies to form calculus, which is commonly called tartar. Tissue destruction, e.g., of periodontal ligament, and alveolar bone resorption can ultimately lead to tooth mobility and subsequent loss of involved teeth.

Peri-implantitis is a destructive inflammatory process affecting the soft and hard tissues surrounding dental implants which is arises due to plaque formation at the tissues surrounding a dental implant.

Thus, in another aspect, the present invention provides a method for preventing gingivitis, periodontitis, and/or peri-implantitis in a subject in need thereof, comprising applying an effective amount of the dental care product of the present invention to a dental enamel surface.

Stannous fluoride has further been clinically demonstrated to significantly reduce dentinal hypersensitivity (Schiff et al., 2005; Hines et al., 2019). Without being bound by theory, the stannous ions from stannous fluoride, in the presence of saliva, forms insoluble tin compounds, i.e., low-solubility hydroxyapatite with tin impurities, that precipitate in the open dentinal tubes and occlude them.

On the other hand, when the herein-described self-assembling peptides self-assemble into a three-dimensional matrix configuration, the matrix forms a stable adherent barrier on exposed dentinal tubules, physically blocking the path of external stimuli.

Thus, the stannous fluoride and the self-assembling peptides present in the dental care product of the present invention promote parallel desensitizing effects. They may act synergistically to reduce dentinal hypersensitivity. Moreover, because the self-assembling peptides in the dental care product also contribute to the maintenance of the stannous ion on the dentin surface, they ensure that the desensitizing effect of stannous fluoride can be prolonged.

Thus, by combining the desensitizing effects of the self-assembling peptides and the stannous fluoride, the dental care product according to the invention provides a more permanent and faster relieve from sensitivity on account of the self-assembling peptide matrix forming an immediate barrier on the exposed dentin. The relief is prolonged by the continued presence of the stannous ion on the root surface through the self-organizing peptide matrix.

Thus, in another aspect, the present invention also provides an improved method for treating dentinal hypersensitivity in a subject in need thereof, comprising applying an effective amount of the dental care product of the present invention to a tooth.

For all the methods described herein, the subject preferably is a human being. It may, however, also be a non-human mammal, e.g., a household pet such as a cat or a dog or another domestic animal such as cow, pig, horse, sheep, goat or camel. The subject may also be another animal kept in captivity such as a non-human primate or, e.g., a lion, tiger or leopard.

In the context of the invention, “treating” any of the above-mentioned conditions is to be understood to comprise a curative medical therapy of a subject with the intent to cure, ameliorate or stabilize said condition. On the other hand, “Preventing” a condition refers to precluding, averting, obviating, forestalling, stopping, or hindering said condition from happening in the first place. Preventing a condition thus also explicitly includes the prophylactic treatment of a subject.

The dental care product may be applied, e.g., by spreading the dental care product using, e.g., a rubber polisher, an applicator such as a syringe, a finger or a toothbrush, as described elsewhere herein.

In the various methods disclosed herein, the dental care product is preferably administered one, two or three times a day on 1, 2, 3, 4, 5, 6, 7 or more days, in one embodiment, daily. It can also be administered less often, e.g., once a week or once a month.

In summary, the present inventors unexpectedly identified a novel way of stabilizing stannous fluoride in a dental care product. By dispersing the stannous fluoride in a three-dimensional matrix formed by self-assembling peptides, it is possible to increase the bioavailabilty of biologically active stannous and fluoride ions. The thus obtained dental care products can be advantageously used in improved methods for treating and/or preventing varies dental conditions, including caries, gingivitis, periodontitis, peri-implantitis and dentinal hypersensitivity.

Throughout the invention, the term “about” is intended to be understood as “+/−10%”. If “about” relates to a range, it refers to both lower and upper limit of the range. “A” is intended to mean “one or more”, if not explicitly mentioned otherwise. The term “comprising” as used herein also encompasses the meaning “consisting of”.

The following examples are intended to illustrate, but not to limit the invention. All literature cited in the present application is herewith incorporated herein in full.

### EXAMPLES

Self-assembling peptides have been proven to enable enamel remineralization, inhibit its demineralization, and treat dentinal hypersensitivity. Stannous fluoride, in a concentration between 0.4-0.5% has also clinically demonstrated efficacy in treating dentinal hypersensitivity and prevention of dental caries.

The inventors made the surprising observation that the combination with the self-assembling peptides considerably increases the stability and thus the bioavailability of the incorporated stannous fluoride, possibly due to its interaction with the self-assembling peptide fibres.

Historically, the use of stannous fluoride in oral care compositions was limited to the problem of instability of the stannous ion (Sn²⁺), which underwent rapid oxidation to the inactive stannic ion (Sn⁴⁺). Intraorally, this also manifests as yellow-brown stains on the tooth surface.

Unexpectedly, this oxidative change is not observed with stannous ions dispersed within the self-assembling peptide matrix. The oxidation is hampered both when exposed to atmospheric oxygen and when tested with redox experiments in the laboratory.

Gel formulations at a pH below 7.5, containing the following, are prepared to evaluate this effect:
1. 0.45% stannous fluoride
2. 0.45% stannous fluoride+50 PPM P11-4 self-assembling peptide matrix
3. 0.45% stannous fluoride+100 PPM P11-4 self-assembling peptide matrix.
4. 0.45% stannous fluoride+250 PPM self-assembling peptide matrix
5. 0.45% stannous fluoride+500 PPM self-assembling peptide matrix

Absence of oxidative changes is observed in all formulations containing 100 PPM or more of self-assembling peptide matrix.

### REFERENCES

White, 1995, A “Return” to stannous fluoride dentifrices. The Journal of Clinical Dentristry. Marquis R E. Antimicrobial actions of fluoride for oral bacteria. Can J Microbiol. 1995 November; 41 (11): 955-64. doi: 10.1139/m95-133. PMID: 7497353.
Nassar Y, Brizuela M. The Role of Fluoride on Caries Prevention. [Updated 2023 Mar. 19]. In: StatPearls [Internet]. Treasure Island (FL): StatPearls Publishing; 2023 January-. Available from: https://www.ncbi.nlm.nih.gov/books/NBK587342/.
https://en.wikipedia.org/wiki/Tin(II)_fluoride
Levine R S. Pyrophosphates in toothpaste: a retrospective and reappraisal. Br Dent J. 2020 November; 229 (10): 687-689. doi: 10.1038/s41415-020-2346-4. Epub 2020 Nov. 27. PMID: 33247264.
Stephen Mason, Sarah Young, Jimmy Qaqish, Guillaume Frappin, Chhaju Goyal, Stain control with two modified stannous fluoride/sodium tripolyphosphate toothpastes: A randomised controlled proof of concept study, Journal of Dentistry, Volume 91, Supplement, 2019.
West N, et al. Erosion protection efficacy of a 0.454% stannous fluoride dentifrice versus an arginine-containing dentifrice. American Journal of Dentistry. 2018 April; 31 (2): 63-66. PMID: 29630787.
Fiorillo L, et al. Stannous Fluoride Effects on Enamel: A Systematic Review. Biomimetics (Basel). 2020 Aug. 31; 5 (3): 41. doi: 10.3390/biomimetics5030041. PMID: 32878006; PMCID: PMC7559150.
Parkinson C R, et al. Gingivitis efficacy of a 0.454% w/w stannous fluoride dentifrice: a 24-week randomized controlled trial. BMC Oral Health. 2020 Mar. 26; 20 (1): 89. doi: 10.1186/s12903-020-01079-6. PMID: 32216778; PMCID: PMC7098169.
Moon J H, et al. Effects of Sodium Tripolyphosphate on Oral Commensal and Pathogenic Bacteria. Pol J Microbiol. 2019; 68 (2): 263-268. doi: 10.33073/pjm-2019-029. PMID: 31257792; PMCID: PMC7256694.
Soares R, De Ataide I N, Fernandes M, Lambor R. Assessment of Enamel Remineralisation After Treatment with Four Different Remineralising Agents: A Scanning Electron Microscopy (SEM) Study. J Clin Diagn Res. 2017 April; 11 (4): ZC136-ZC141. doi: 10.7860/JCDR/2017/23594.9758. Epub 2017 Apr. 1. PMID: 28571281; PMCID: PMC5449906.
Jablonsky-Momeni et al., 2019. Randomised in situ clinical trial investigating self-assembling peptide matrix P11-4 in the prevention of artificial caries lesions. Scientific Reports 9:269.
Acherkouk A et al. A randomised clinical study investigating efficacy of a stannous fluoride toothpaste in improving gingival health after 3 weeks' use. BMC Oral Health. 2021 Sep. 12; 21 (1): 441. doi: 10.1186/s12903-021-01727-5. PMID: 34511098; PMCID: PMC8436562.
Paraskevas S, van der Weijden G A. A review of the effects of stannous fluoride on gingivitis. J Clin Periodontol. 2006 January; 33 (1): 1-13. doi: 10.1111/j. 1600-051X.2005.00860.x. PMID: 16367849.
Schiff T, et al. Desensitizing effect of a stabilized stannous fluoride/Sodium hexametaphosphate dentifrice. Compend Contin Educ Dent. 2005 September; 26 (9 Suppl 1): 35-40. PMID: 16999008.
Hines D, et al. Effect of a stannous fluoride toothpaste on dentinal hypersensitivity: In vitro and clinical evaluation. J Am Dent Assoc. 2019 April; 150 (4S): S47-S59. doi: 10.1016/j.adaj.2019.01.006. PMID: 30797259.
U.S. Pat. No. 4,418,057A
U.S. Pat. No. 3,433,544A
U.S. Pat. No. 5,004,597A
U.S. Pat. No. 5,716,600A
U.S. Pat. No. 5,004,597A
U.S. Pat. No. 9,968,803B2
U.S. Pat. No. 3,282,792A
U.S. Pat. No. 5,017,363A
U.S. Pat. No. 4,960,586A
U.S. Pat. No. 4,961,924A
WO 2004/007532 A1
U.S. Ser. No. 10/521,628
U.S. Ser. No. 12/729,046
U.S. Ser. No. 13/551,878
U.S. Ser. No. 14/062,768
WO 2010/041636 A1
WO2014/027012 A1
U.S. Pat. No. 6,667,027B2
U.S. Pat. No. 6,350,436B1
U.S. Pat. No. 6,821,507B2
U.S. Pat. No. 5,578,293A
U.S. Pat. No. 5,145,666A
U.S. Pat. No. 5,281,411A
U.S. Pat. No. 5,281,410A
WO 2021/110923 A1

1. A dental care product comprising:
i. tin(II) fluoride, and
ii. at least 0.01 wt % self-assembling peptides comprising a sequence of X1-X2-X1-X2-X1, wherein X1 is independently selected from the group consisting of glutamic acid, aspartic acid, glutamine and ornithine, and X2 is independently selected from the group consisting of alanine, valine, isoleucine, leucine, methionine, phenylalanine, tyrosine, tryptophan and glutamine, wherein the self-assembling peptides are essentially present in the dental care product in assembled form.

2. The dental care product of claim 1, wherein the tin(II) fluoride is present in the dental care product in a concentration of 0.1-1.0 wt %.

3. The dental care product of claim 1, wherein the self-assembling peptides comprise an amino acid sequence of SEQ ID NO: 6 (P11-4) or SEQ ID NO: 9 (P11-8) or SEQ ID NO: 15 (P11-20) or consist thereof.

4. The dental care product of claim 1, wherein the self-assembling peptides consist of SEQ ID NO: 6 (P11-4).

5. The dental care product of claim 1, further comprising a polyphosphate.

6. The dental care product of claim 5, wherein the polyphosphate is sodium tripolyphosphate.

7. The dental care product of claim 6, wherein the sodium tripolyphosphate is present in the dental care product at a concentration of 1-10 wt %.

8. The dental care product of claim 1 further comprising sodium gluconate.

9. The dental care product of claim 8, wherein the sodium gluconate is present in the dental care product at a concentration of 0.5-5 wt %.

10. The dental care product of claim 1, wherein the dental care product is a dental gel, a toothpaste, a prophylactic paste, a tooth foam or a dental rinse.

11. The dental care product of claim 1, further comprising
a. sodium tripolyphosphate,
b. sodium gluconate,
C. sorbitol,
d. cellulose gum,
e. xylitol,
f. erythritol,
g. hydrated silica,
h. phosphoric acid,
i. optionally, NaOH, and
j. water

12. The dental care product of claim 1, wherein the pH of the dental care product is below 7.5.

13. A dental care product comprising:
i. 0.1-1.0 wt % tin(II) fluoride;
ii. at least 0.01 wt % self-assembling peptides consisting of SEQ ID NO: 6 (P11-4);
iii. 1-10 wt % sodium tripolyphosphate; and
iv. 0.5-5 wt % sodium gluconate,
wherein the pH of the dental care product is below 7.5.

14. A method for preventing demineralization of a tooth, comprising applying an effective amount of the dental care product of claim 1 to the tooth.

15. A method for remineralizing tooth enamel in a subject in need thereof, comprising applying an effective amount of the dental care product of claim 1 to a tooth.

16. A method for destroying biofilm formed on a tooth, comprising applying an effective amount of the dental care product of claim 1 to a tooth.

17. A method for treating dentinal hypersensitivity in a subject in need thereof, comprising applying an effective amount of the dental care product of claim 1 to a tooth.

18. A method for stabilizing stannous fluoride in a dental care product, comprising admixing
i) tin(II) fluoride, and
i) at least 0.01 wt % self-assembling peptides comprising a sequence of X1-X2-X1-X2-X1, wherein X1 is independently selected from the group consisting of glutamic acid, aspartic acid, glutamine and ornithine, and X2 is independently selected from the group consisting of alanine, valine, isoleucine, leucine, methionine, phenylalanine, tyrosine, tryptophan and glutamine, wherein the self-assembling peptides are essentially present in the dental care product in assembled form.


SEQUENCE LISTING (21 sequences)

SEQ ID NO: 1-4 - skipped (4 entries)

SEQ ID NO: 5 - protein, 11 aa, synthetic construct
  SITE 5: note = independently selected from the group consisting of glutamic acid and ornithine
  SITE 7: note = independently selected from the group consisting of glutamic acid and ornithine
  SITE 9: note = independently selected from the group consisting of glutamic acid and ornithine
QQRFXWXFXQQ

SEQ ID NO: 6 - protein, 11 aa, synthetic construct
QQRFEWEFEQQ

SEQ ID NO: 7 - protein, 11 aa, synthetic construct
QQRFQWQFEQQ

SEQ ID NO: 8 - protein, 11 aa, synthetic construct
  SITE 5: note = ornithine
  SITE 7: note = ornithine
QQRFXWXFQQQ

SEQ ID NO: 9 - protein, 11 aa, synthetic construct
  SITE 5: note = ornithine
  SITE 7: note = ornithine
QQRFXWXFEQQ

SEQ ID NO: 10 - protein, 11 aa, synthetic construct
  SITE 5: note = ornithine
  SITE 7: note = ornithine
SSRFXWXFESS

SEQ ID NO: 11 - protein, 11 aa, synthetic construct
EQEFEWEFEQE

SEQ ID NO: 12 - protein, 11 aa, synthetic construct
  SITE 3: note = ornithine
  SITE 5: note = ornithine
  SITE 7: note = ornithine
  SITE 9: note = ornithine
QQXFXWXFXQQ

SEQ ID NO: 13 - protein, 11 aa, synthetic construct
TTRFEWEFETT

SEQ ID NO: 14 - protein, 11 aa, synthetic construct
  SITE 5: note = ornithine
  SITE 7: note = ornithine
QQRQXQXQEQQ

SEQ ID NO: 15 - protein, 11 aa, synthetic construct
QQRQEQEQEQQ

SEQ ID NO: 16 - protein, 11 aa, synthetic construct
  SITE 1: note = ornithine
  SITE 3: note = ornithine
  SITE 5: note = ornithine
  SITE 7: note = ornithine
  SITE 9: note = ornithine
  SITE 11: note = ornithine
XQXFXWXFXQX

SEQ ID NO: 17 - protein, 11 aa, synthetic construct
QQEFEWEFEQQ

SEQ ID NO: 18 - protein, 11 aa, synthetic construct
  SITE 5: note = ornithine
  SITE 7: note = ornithine
NNRFXWXFENN

SEQ ID NO: 19 - protein, 11 aa, synthetic construct
  SITE 5: note = ornithine
  SITE 7: note = ornithine
TTRFXWXFETT

SEQ ID NO: 20 - protein, 11 aa, synthetic construct
  SITE 3: note = ornithine
  SITE 5: note = ornithine
  SITE 7: note = ornithine
  SITE 9: note = ornithine
QQXQXQXQXQQ

SEQ ID NO: 21 - protein, 11 aa, synthetic construct
  SITE 3: note = ornithine
  SITE 5: note = ornithine
  SITE 7: note = ornithine
  SITE 9: note = ornithine
SSXFXWXFXSS